(19) United States
(12) Patent Application Publication
(10) Pub. No.: US 2021/0038616 A1
(43) Pub. Date: Feb. 11, 2021

(54) SOLID OILY EXTERNAL COMPOSITION

(71) Applicant: ROHTO PHARMACEUTICAL CO., LTD., Osaka-shi Osaka (JP)

(72) Inventors: Noriko OHTA, Osaka-shi Osaka (JP); Junko MARUKAWA, Osaka-shi Osaka (JP)

(73) Assignee: ROHTO PHARMACEUTICAL CO., LTD., Osaka-shi Osaka (JP)

(21) Appl. No.: 16/979,757

(22) PCT Filed: Mar. 29, 2019
(86) PCT No.: PCT/JP2019/013988

§ 371 Date: Sep. 10, 2020

(30) Foreign Application Priority Data
Mar. 30, 2018 (JP) 2018-068952

(51) Int. Cl. A61K 31/573; A61K 47/06; A61K 9/00; A61K 31/135

(52) U.S. Cl. CPC A61K 31/573; A61K 31/135; A61K 9/0014; A61K 47/06

(57) ABSTRACT

A solid oily external composition including at least one selected from the group consisting of (A) a steroid compound and an antihistamine is smoothly applicable onto a skin or a mucous membrane. The composition can be a stick-shaped solid composition. The content of the steroid compound (A) can be 0.0001 to 5% by weight relative to the total amount of the composition, and the content of the antihistamine (B) can be 0.01 to 5% by weight relative to the total amount of the composition.

### TECHNICAL FIELD

The present invention relates to an oily external composition having a solid shape such as a stick shape.

### BACKGROUND ART

As a cosmetic or a quasi-drug, such as a lipstick, a lip cream, a foundation, a makeup base, an eye cream, an antiperspirant, and a whitening agent, an oily composition molded into a stick shape or the like has been widely used. Such a solid composition has the following advantages: the composition can be simply used not to make hands dirty when the composition is applied; and the composition can be accurately applied onto a narrow area.

However, such a solid oily composition is difficult to spread when applied onto a skin or a mucous membrane such as lips because of stickiness to the skin or the mucous membrane. In recent years, consumers value sense of use, and thus a composition to be commercialized is required to be smoothly applicable onto a skin or a mucous membrane.

### SUMMARY OF INVENTION

### Technical Problem

The present invention is intended to provide a solid oily external composition smoothly applicable onto a skin or a mucous membrane.

### Solution to Problem

The inventors of the present invention have conducted intensive studies in order to solve the above problem and have found that solid oily compositions have various smoothnesses at the time of application depending on not only formulations of the base material but also ingredients such as an active ingredient and additives. The inventors of the present invention have further found that a solid oily composition particularly containing (A) a steroid compound and/or (B) an antihistamine has smaller friction at the time of application onto a skin or a mucous membrane and can be smoothly applied.

The present invention has been completed on the basis of the above findings and provides the following solid oily external compositions.

Item 1: A solid oily external composition including at least one selected from the group consisting of (A) a steroid compound and (B) an antihistamine.

Item 2: The composition according to item 1, in which the steroid compound (A) is a compound selected from the group consisting of prednisolone, dexamethasone, hydrocortisone, esters thereof, and salts thereof.

Item 3: The composition according to item 2, in which the steroid compound (A) is a compound selected from the group consisting of prednisolone valerate acetate, dexamethasone acetate, hydrocortisone, and hydrocortisone acetate.

Item 4: The composition according to any one of items 1 to 3, in which the antihistamine (B) is at least one selected from the group consisting of diphenhydramine and salts thereof.

Item 5: The composition according to any one of items 1 to 4, in which the steroid compound (A) is contained at a content of 0.0001 to 5% by weight relative to a total amount of the composition.

Item 6: The composition according to any one of items 1 to 5, in which the antihistamine (B) is contained at a content of 0.01 to 5% by weight relative to a total amount of the composition.

Item 7: The composition according to anyone of items 1 to 6, further including at least one selected from the group consisting of (C) a local anesthetic, (D) an antipruritic other than the antihistamine, (E) an anti-inflammatory agent, (F) a sterilizer, and (G) a refreshing agent.

Item 8: The composition according to any one of items 1 to 7, in which the composition is a skin external composition.

Item 9: The composition according to any one of items 1 to 8, in which the composition has a stick shape.

Item 10: A method of reducing a friction of a solid oily external composition against a skin or a mucous membrane when the composition is applied, the method including adding (A) a steroid compound and/or (B) an antihistamine to the composition.

### Advantageous Effects of Invention

Generally, a typical solid oily composition has a large friction and is difficult to spread when applied onto a skin or a mucous membrane. In contrast, the solid oily external composition of the present invention contains a steroid compound and/or an antihistamine and thus can be smoothly applied onto a skin or a mucous membrane.

As described above, the solid oily external composition of the present invention has excellent sense of use at the time of application and accordingly has a high commercial value. If a composition has a poor smoothness at the time of application, the composition may cause irritation when an application area has inflammation or damage, or scraped tissues of a skin or lips or dusts may adhere to the composition, which is likely to lead to poor hygiene. The solid oily external composition of the present invention has a small friction against a skin or a mucous membrane and thus is easily applied onto an area having inflammation or damage, and scraped tissues or dusts are unlikely to adhere to the composition. The above ingredients have pharmacological activities and thus are preferably applied onto an affected area in a limited manner and onto an area other than the affected area in a smaller amount. The solid oily external composition of the present invention can be smoothly applied and thus is easily applied accurately onto an intended area.

### BRIEF DESCRIPTION OF DRAWINGS

FIG. 1 is a graph showing that addition of (A) a steroid compound and/or (B) an antihistamine to a solid oily composition reduced friction coefficients against a skin.

### DESCRIPTION OF EMBODIMENTS

The present invention will now be described in detail.

The composition of the present invention is a solid oily external composition including (A) a steroid compound and/or (B) an antihistamine.

### (A) Steroid Compound

Specific examples of the steroid compound include prednisolone, hydrocortisone, cortisone, dexamethasone, triamcinolone, triamcinolone acetonide, difluprednate, mometasone, diflucortolone, fluocinonide, beclomethasone, deprodone, alclometasone, flumethasone, amcinonide, clobetasone, diflorasone, and derivatives thereof.

Examples of the derivative typically includes esters (especially, esters with an organic acid or an inorganic acid), salts, and salts of esters.

The steroid compound can be a hydrate, a hemihydrate, or an anhydride.

Examples of the ester include an ester with an organic acid such as valeric acid, acetic acid, succinic acid, butyric acid (butanoic acid), propionic acid, sulfobenzoic acid, cipecilic acid, palmitic acid, furancarboxylic acid, and pivalic acid; and an ester with an inorganic acid such as phosphoric acid. The steroid compound may contain a plurality of ester groups per molecule. A steroid compound containing a plurality of ester groups per molecule may contain a plurality of ester groups with a single acid or may contain ester groups with two or more acids.

Examples of the salt include a salt with an organic base (including organic amine salts such as a methylamine salt, a triethylamine salt, a triethanolamine salt, a morpholine salt, a piperazine salt, a pyrrolidine salt, a tripyridine salt, and a picoline salt) and a salt with an inorganic base (including an ammonium salt; alkali metal salts such as a sodium salt and a potassium salt; alkaline earth metal salts such as a calcium, salt and a magnesium salt; and metal salts such as a zinc salt and an aluminum salt). Of them, an alkali metal salt such as a sodium salt and a potassium salt is preferred.

Specific examples of the steroid compound derivative include prednisolone esters such as prednisolone valerate acetate (PVA), prednisolone succinate, prednisolone acetate, and prednisolone phosphate, dexamethasone esters such as dexamethasone valerate, dexamethasone propionate, dexamethasone acetate, dexamethasone phosphate, dexamethasone metasulfobenzoate, dexamethasone cipecilate, and dexamethasone palmitate, hydrocortisone esters such as hydrocortisone butyrate (specifically, hydrocortisone-17-butyrate), hydrocortisone acetate, hydrocortisone succinate, hydrocortisone butyrate, hydrocortisone butyrate propionate, and hydrocortisone phosphate, mometasone furoate, diflucortolone valerate, beclomethasone propionate, beclomethasone dipropionate, clobetasone butyrate, deprodone propionate, alclometasone propionate, flumethasone pivalate, clobetasone propionate, clobetasone butyrate, and diflorasone acetate.

Of them, prednisolone valerate acetate, dexamethasone acetate, hydrocortisone, and hydrocortisone acetate are preferred, prednisolone valerate acetate, hydrocortisone, and hydrocortisone acetate are more preferred, and prednisolone valerate acetate is particularly preferred.

When no antihistamine is contained, use of prednisolone, dexamethasone, hydrocortisone, and/or a derivative thereof (especially, an ester) as the steroid compound markedly reduces the friction against a skin. Specifically, prednisolone valerate acetate, dexamethasone acetate, hydrocortisone, and hydrocortisone acetate are preferred, prednisolone valerate acetate, hydrocortisone, and hydrocortisone acetate are more preferred, and prednisolone valerate acetate is particularly preferred.

The steroid compounds may be used singly or in combination of two or more of them.

The content of the steroid compound is preferably 0.0001% by weight or more, more preferably 0.001% by weight or more, and even more preferably 0.01% by weight or more relative to the total amount of the composition. The content is preferably 5% by weight or less, more preferably 2.5% by weight or less, even more preferably 1% by weight or less, and still more preferably 0.5% by weight or less. A composition having such a content as above achieves an appropriate pharmacological activity and has satisfactory smoothness.

The content of the steroid compound relative to the total amount of the composition is 0.0001 to 5% by weight, 0.0001 to 2.5% by weight, 0.0001 to 1% by weight, 0.0001 to 0.5% by weight, 0.001 to 5% by weight, 0.001 to 2.5% by weight, 0.001 to 1% by weight, 0.001 to 0.5% by weight, 0.01 to 5% by weight, 0.01 to 2.5% by weight, 0.01 to 1% by weight, or 0.01 to 0.5% by weight, for example.

Specifically, the content of prednisolone or a derivative thereof (especially, an ester) is preferably 0.01% by weight or more, more preferably 0.06% by weight or more, and even more preferably 0.1% by weight or more relative to the total amount of the composition. The content is preferably 1% by weight or less, more preferably 0.5% by weight or less, and even more preferably 0.2% by weight or less. The content is particularly preferably 0.15% by weight.

The content of the prednisolone or a derivative thereof (especially, an ester) relative to the total amount of the composition is 0.01 to 1% by weight, 0.01 to 0.5% by weight, 0.01 to 0.2% by weight, 0.01 to 0.15% by weight, 0.06 to 1% by weight, 0.06 to 0.5% by weight, 0.06 to 0.2% by weight, 0.06 to 0.15% by weight, 0.1 to 1% by weight, 0.1 to 0.5% by weight, 0.1 to 0.2% by weight, or 0.1 to 0.15% by weight, for example.

Specifically, the content of dexamethasone or a derivative thereof (especially, an ester) is preferably 0.005% by weight or more, more preferably 0.01% by weight or more, and even more preferably 0.0125% by weight or more relative to the total amount of the composition. The content is preferably 0.1% by weight or less, more preferably 0.05% by weight or less, and even more preferably 0.025% by weight or less.

The content of dexamethasone or a derivative thereof (especially, an ester) relative to the total amount of the composition is 0.005 to 0.1% by weight, 0.005 to 0.05% by weight, 0.005 to 0.025% by weight, 0.01 to 0.1% by weight, 0.01 to 0.05% by weight, 0.01 to 0.025% by weight, 0.0125 to 0.1% by weight, 0.0125 to 0.05% by weight, or 0.0125 to 0.025% by weight, for example.

Specifically, the content of hydrocortisone or a derivative thereof (especially, an ester) is preferably 0.05% by weight or more, more preferably 0.1% by weight or more, and even more preferably 0.15% by weight or more relative to the total amount of the composition. The content is preferably 2% by weight or less, more preferably 1% by weight or less, and even more preferably 0.5% by weight or less.

The content of hydrocortisone or a derivative thereof (especially, an ester) relative to the total amount of the composition is 0.05 to 2% by weight, 0.05 to 1% by weight, 0.05 to 0.5% by weight, 0.1 to 2% by weight, 0.1 to 1% by weight, 0.1 to 0.5% by weight, 0.15 to 2% by weight, 0.15 to 1% by weight, or 0.15 to 0.5% by weight, for example.

### (B) Antihistamine

Examples of the antihistamine include ethanolamine antihistamines such as diphenhydramine, bromodiphenhydramine, clemastine, chlorphenoxamine, diphenylpyraline, doxylamine, orphenadrine, and phenyltoloxamine, propylamine antihistamines such as chlorpheniramine, dimethindene, and talastine, ethylenediamine antihistamines such as mepyramine, methapyrilene, and tripelennamine, phenothiazine antihistamines such as alimemazine, hydroxyethylpromethazine, isothipendyl, mequitazine, oxomemazine, and promethazine, piperazine antihistamines such as buclizine, cetirizine, homochlorcyclizine, cyclizine, hydroxyzine, levocetirizine, meclizine, and oxatomide, and ketotifen, olopatadine, fexofenadine, loratadine, terfenadine, antazoline, azatadine, bamipine, cyproheptadine, deptropine, ebastine, emedastine, epinastine, mebhydroline, mizolastine, pimethixene, pyrrobutamine, quifenadine, rupatadine, triprolidine, acrivastine, astemizole, azelastine, bilastine, desloratadine, and salts thereof.

The antihistamine can be a hydrate, a hemihydrate, or an anhydride.

The salt can be a pharmaceutically or physiologically acceptable salt, and examples include inorganic acid salts and organic acid salts. Examples of the inorganic acid salt include a hydrochloride, a hydrobromide, a nitrate, a sulfate, and a phosphate. Examples of the organic acid salt include monocarboxylates such as an acetate, a trifluoroacetate, a butyrate, a palmitate, and a stearate, polycarboxylates such as a fumarate, a maleate, a succinate, and a malonate, oxycarboxylates such as a lactate, a tartrate, and a citrate, and organic sulfonates such as a methanesulfonate, a toluenesulfonate, a tosylate, and a napadisilate.

Specific examples include diphenhydramine hydrochloride, bromodiphenhydramine hydrochloride, clemastine fumarate, chlorphenoxamine hydrochloride, diphenylpyraline hydrochloride, doxylamine succinate, orphenadrine hydrochloride, phenyltoloxamine citrate, chlorpheniramine maleate, dimethindene maleate, mepyramine maleate, methapyrilene hydrochloride, tripelennamine hydrochloride, alimemazine tartrate, isothipendyl hydrochloride, oxomemazine hydrochloride, promethazine hydrochloride, cetirizine hydrochloride, homochlorcyclizine hydrochloride, cyclizine hydrochloride, hydroxyzine hydrochloride, levocetirizine hydrochloride, meclizine hydrochloride, ketotifen fumarate, olopatadine hydrochloride, fexofenadine hydrochloride, antazoline hydrochloride, azatadine dimaleate, bamipine hydrochloride, cyproheptadine hydrochloride, deptropine citrate, emedastine fumarate, epinastine hydrochloride, mebhydroline napadisilate, rupatadine fumarate, triprolidine hydrochloride, and azelastine hydrochloride.

Of them, ethanolamine antihistamines are preferred, diphenhydramine or a salt thereof (especially, a hydrochloride) is more preferred, and diphenhydramine is even more preferred.

The antihistamines may be used singly or in combination of two or more of them.

The content of the antihistamine is preferably 0.01% by weight or more, more preferably 0.1% by weight or more, and even more preferably 0.5% by weight or more relative to the total amount of the composition. The content is preferably 5% by weight or less, more preferably 3% by weight or less, and even more preferably 2% by weight or less. The content can be 1% by weight or less. A composition having such a content as above achieves an appropriate pharmacological activity and has satisfactory smoothness.

The content of the antihistamine relative to the total amount of the composition is 0.01 to 5% by weight, 0.01 to 3% by weight, 0.01 to 2% by weight, 0.01 to 1% by weight, 0.1 to 5% by weight, 0.1 to 3% by weight, 0.1 to 2% by weight, 0.1 to 1% by weight, 0.5 to 5% by weight, 0.5 to 3% by weight, 0.5 to 2% by weight, or 0.5 to 1% by weight, for example.

The solid oily external composition of the present invention has such an effect as to be applicable smoothly even when containing, in addition to the ingredient (A) and/or the ingredient (B), one or two or more of (C) an antipruritic other than the antihistamine, (D) a local anesthetic, (E) a non-steroidal anti-inflammatory agent, (F) a sterilizer, and (G) a refreshing agent. The “two or more” encompasses a case containing the ingredient (C) and the ingredient (D) or a case containing two or more of the ingredients (C), for example.

(C) Antipruritic Other than Antihistamine

Examples of the antipruritic other than the antihistamine include crotamiton, ichthammol, wood tar, and thymol. Of them, crotamiton is preferred.

The antipruritic other than the antihistamine can be a hydrate, a hemihydrate, or an anhydride.

The content of the antipruritic other than the antihistamine is preferably 0.01% by weight or more, more preferably 0.1% by weight or more, and even more preferably 1% by weight or more relative to the total amount of the composition. The content is preferably 20% by weight or less, more preferably 15% by weight or less, and even more preferably 10% by weight or less. A composition having such a content as above achieves an appropriate pharmacological activity and has satisfactory smoothness.

The content of the antipruritic other than the antihistamine relative to the total amount of the composition is 0.01 to 20% by weight, 0.01 to 15% by weight, 0.01 to 10% by weight, 0.1 to 20% by weight, 0.1 to 15% by weight, 0.1 to 10% by weight, 1 to 20% by weight, 1 to 15% by weight, or 1 to 10% by weight, for example.

### (D) Local Anesthetic

Examples of the local anesthetic include local anesthetics having an amine structure and an amide structure, such as lidocaine, dibucaine, mepivacaine, bupivacaine, ropivacaine, levobupivacaine, oxethazaine, and salts thereof, local anesthetics having an amine structure and an ester structure, such as cocaine, procaine, chloroprocaine, tetracaine, and salts thereof, ethyl aminobenzoate having an ester structure, and oxypolyethoxydodecane.

The local anesthetic can be a hydrate, a hemihydrate, or an anhydride.

The salt can be a pharmaceutically or physiologically acceptable salt, and examples include inorganic acid salts and organic acid salts. Examples of the inorganic acid salt include a hydrochloride, a hydrobromide, a nitrate, a sulfate, and a phosphate. Examples of the organic acid salt include monocarboxylates such as an acetate, a trifluoroacetate, a butyrate, a palmitate, and a stearate, polycarboxylates such as a fumarate, a maleate, a succinate, and a malonate, oxycarboxylates such as a lactate, a tartrate, and a citrate, and organic sulfonates such as a methanesulfonate, a toluenesulfonate, a tosylate, and a napadisilate.

Specific examples include lidocaine hydrochloride, dibucaine hydrochloride, mepivacaine hydrochloride, bupivacaine hydrochloride, ropivacaine hydrochloride, levobupivacaine hydrochloride, oxethazaine hydrochloride, cocaine hydrochloride, procaine hydrochloride, chloroprocaine hydrochloride, and tetracaine hydrochloride.

Of them, lidocaine, dibucaine, ethyl aminobenzoate, oxypolyethoxydodecane, or a salt thereof (a hydrochloride and the like) is preferred, lidocaine or a salt thereof is more preferred, lidocaine or lidocaine hydrochloride is even more preferred, and lidocaine is still more preferred.

The local anesthetics may be used singly or in combination of two or more of them.

The content of the local anesthetic is preferably 0.01% by weight or more, more preferably 0.05% by weight or more, even more preferably 0.1% by weight or more, and still more preferably 0.5% by weight or more relative to the total amount of the composition. The content can be 1% by weight or more. The content is preferably 5% by weight or less, more preferably 3% by weight or less, and even more preferably 2% by weight or less. A composition having such a content as above achieves an appropriate pharmacological activity and has satisfactory smoothness.

The content of the local anesthetic relative to the total amount of the composition is 0.01 to 5% by weight, 0.01 to 3% by weight, 0.01 to 2% by weight, 0.05 to 5% by weight, 0.05 to 3% by weight, 0.05 to 2% by weight, 0.1 to 5% by weight, 0.1 to 3% by weight, 0.1 to 2% by weight, 0.5 to 5% by weight, 0.5 to 3% by weight, 0.5 to 2% by weight, 1 to 5% by weight, 1 to 3% by weight, or 1 to 2% by weight, for example.

### (E) Non-Steroidal Anti-Inflammatory Agent

Examples of the non-steroidal anti-inflammatory agent include allantoin, glycyrrhizic acid, methyl glycyrrhizinate, stearyl glycyrrhizinate, glycyrrhetinic acid, stearyl glycyrrhetinate, acetaminophen, epsilon-aminocaproic acid, berberine, azulene, bromelain, and zinc; plant extracts such as glycyrrhiza extract, sage extract, and rosemary extract; enzyme anti-inflammatory agents such as lysozyme, serrapeptase, and semi-alkaline proteinase; fenamic acid anti-inflammatory agents such as mefenamic acid, flufenamic acid, and tolfenamic acid; arylacetic acid anti-inflammatory agents such as acemetacin, indomethacin, indomethacin farnesil, etodolac, diclofenac, sulindac, nabumetone, fenbufen, proglumetacin, and mofezolac; propionic acid anti-inflammatory agents such as alminoprofen, ibuprofen, oxaprozin, ketoprofen, zaltoprofen, tiaprofenic acid, naproxen, flurbiprofen, zaltoprofen, ibuprofen piconol, flurbiprofen axetil, fenoprofen, pranoprofen, and loxoprofen; and oxicam anti-inflammatory agents such as ampiroxicam, tenoxicam, piroxicam, meloxicam, and lornoxicam. These compounds can be a salt.

The non-steroidal anti-inflammatory agent can be a hydrate, a hemihydrate, or an anhydride.

The salt can be a pharmaceutically or physiologically acceptable salt, and examples include inorganic acid salts and organic acid salts. Examples of the inorganic acid salt include a hydrochloride, a hydrobromide, a nitrate, a sulfate, and a phosphate. Examples of the organic acid salt include monocarboxylates such as an acetate, a trifluoroacetate, a butyrate, a palmitate, and a stearate, polycarboxylates such as a fumarate, a maleate, a succinate, and a malonate, oxycarboxylates such as a lactate, a tartrate, and a citrate, and organic sulfonates such as a methanesulfonate, a toluenesulfonate, a tosylate, and a napadisilate.

Examples of the salt also include a salt with an organic base (including organic amine salts such as a methylamine salt, a triethylamine salt, a triethanolamine salt, a morpholine salt, a piperazine salt, a pyrrolidine salt, a tripyridine salt, and a picoline salt) and a salt with an inorganic base (including an ammonium salt; alkali metal salts such as a sodium salt and a potassium salt; alkaline earth metal salts such as a calcium salt and a magnesium salt; and metal salts such as a zinc salt and an aluminum salt).

Specific examples include allantoin, dipotassium glycyrrhizinate, monoammonium glycyrrhizinate, glycyrrhetinic acid, stearyl glycyrrhetinate, indomethacin hydrochloride, diclofenac sodium, bromfenac sodium, berberine sulfate, berberine hydrochloride, berberine tannate, sodium azulene sulfonate, zinc sulfate, zinc lactate, lysozyme hydrochloride, proglumetacin maleate, fenoprofen calcium, and loxoprofen sodium.

Of them, allantoin, dipotassium glycyrrhizinate, monoammonium glycyrrhizinate, glycyrrhetinic acid, and stearyl glycyrrhetinate are preferred, allantoin, dipotassium glycyrrhizinate, monoammonium glycyrrhizinate, and glycyrrhetinic acid are more preferred, and glycyrrhetinic acid is even more preferred.

The non-steroidal anti-inflammatory agents may be used singly or in combination of two or more of them.

The content of the non-steroidal anti-inflammatory agent is preferably 0.01% by weight or more, more preferably 0.1% by weight or more, and even more preferably 0.5% by weight or more relative to the total amount of the composition. The content is preferably 5% by weight or less, more preferably 3% by weight or less, and even more preferably 1% by weight or less. A composition having such a content as above achieves an appropriate pharmacological activity and has satisfactory smoothness.

The content of the non-steroidal anti-inflammatory agent relative to the total amount of the composition is 0.01 to 5% by weight, 0.01 to 3% by weight, 0.01 to 1% by weight, 0.1 to 5% by weight, 0.1 to 3% by weight, 0.1 to 1% by weight, 0.5 to 5% by weight, 0.5 to 3% by weight, or 0.5 to 1% by weight, for example.

### (F) Sterilizer

Examples of the sterilizer include isopropylmethylphenol, phenoxyethanol, dequalinium chloride, benzalkonium chloride, benzethonium chloride, chlorhexidine hydrochloride, chlorhexidine gluconate, alkyldiaminoethylglycine hydrochloride, cetylpyridinium chloride, sodium benzoate, ethanol, chlorobutanol, sorbic acid, potassium sorbate, sodium dehydroacetate, methyl p-hydroxybenzoate, ethyl p-hydroxybenzoate, propyl p-hydroxybenzoate, butyl p-hydroxybenzoate, oxyquinoline sulfate, phenethyl alcohol, benzyl alcohol, salicylic acid, cresol, triclosan, and biguanide compounds.

Of them, isopropylmethylphenol, benzalkonium chloride, and benzethonium chloride are preferred, and isopropylmethylphenol is more preferred.

The sterilizers may be used singly or in combination of two or more of them.

The sterilizer can be a hydrate, a hemihydrate, or an anhydride.

In the description, the solid oily external composition of the present invention does not necessarily contain antibiotics.

The content of the sterilizer is preferably 0.001% by weight or more, more preferably 0.01% by weight or more, and even more preferably 0.05% by weight or more relative to the total amount of the composition. The content is preferably 3% by weight or less, more preferably 1% by weight or less, and even more preferably 0.5% by weight or less. A composition having such a content as above achieves an appropriate sterilization effect and has satisfactory smoothness.

The content of the sterilizer relative to the total amount of the composition is 0.001 to 3% by weight, 0.001 to 1% by weight, 0.001 to 0.5% by weight, 0.01 to 3% by weight, 0.01 to 1% by weight, 0.01 to 0.5% by weight, 0.05 to 3% by weight, 0.05 to 1% by weight, or 0.05 to 0.5% by weight, for example.

### (G) Refreshing Agent

Examples of the refreshing agent include terpenes such as menthol, camphor, borneol, geraniol, cineole, anethole, limonene, and eugenol (including d-forms, l-forms, and dl-forms thereof); and essential oils such as eucalyptus oil, bergamot oil, peppermint oil, cool mint oil, spearmint oil, fennel oil, Japanese mint oil, cinnamon oil, rose oil, and turpentine oil.

Of them, menthol, camphor, and borneol are preferred, and menthol is more preferred.

The refreshing agents may be used singly or in combination of two or more of them.

The content of the refreshing agent is preferably 0.001% by weight or more, more preferably 0.01% by weight or more, and even more preferably 0.1% by weight or more relative to the total amount of the composition. The content is preferably 10% by weight or less, more preferably 5% by weight or less, and even more preferably 3.5% by weight or less. A composition having such a content as above achieves an appropriate refreshing effect and has satisfactory smoothness.

The content of the refreshing agent relative to the total amount of the composition is 0.001 to 10% by weight, 0.001 to 5% by weight, 0.001 to 3.5% by weight, 0.01 to 10% by weight, 0.01 to 5% by weight, 0.01 to 3.5% by weight, 0.1 to 10% by weight, 0.1 to 5% by weight, or 0.1 to 3.5% by weight, for example.

### Other Ingredients

The solid oily external composition of the present invention can be an external composition of a pharmaceutical product, a quasi-drug, or a cosmetic prepared by mixing the above active ingredients with a base material or carrier used in a pharmaceutical product, a quasi-drug, or a cosmetic and, as needed, with additives or additional physiologically active or pharmacologically active ingredients. In particular, the composition can be a pharmaceutical composition (pharmaceutical external composition).

Examples of the additive include an antioxidant, a surfactant, a thickener, an antiseptic or preservative, a ph adjuster, a stabilizer, a chelating agent, an ultraviolet absorber or ultraviolet scattering agent, an irritation reducing agent, a coloring agent, and a flavoring agent.

The additives may be used singly or in combination of two or more of them.

The additive can be used in such a range as not to impair the effect of the invention.

Examples of the antioxidant include dibutylated hydroxytoluene, butylated hydroxyanisole, p-hydroxyanisole, sorbic acid, sodium sulfite, ascorbic acid, ascorbic acid derivatives (such as ascorbyl stearate, ascorbyl palmitate, ascorbyl dipalmitate, ascorbyl monophosphate, ascorbyl diphosphate, ascorbyl triphosphate, and ascorbyl sulfate), tocopherol, tocopherol derivatives (such as tocopherol acetate, tocopherol succinate, and tocopherol calcium, succinate), erythorbic acid, L-cysteine hydrochloride, lycopene, glutathione, propyl gallate, tannic acid, epigallocatechin, anthocyanin, hydroxytyrosol, nordihydroguaiaretic acid, caffeic acid, and enzymes (such as catalase, superoxide dismutase, glutathione peroxidase, and elastase).

When the composition of the present invention contains an aqueous base material such as water or a water-soluble ingredient, a surfactant may be mixed.

Examples of the surfactant include sorbitan fatty acid esters such as sorbitan monoisostearate, sorbitan monolaurate, sorbitan monopalmitate, sorbitan monostearate, diglycerol sorbitan pent-2-ethylhexylate, and diglycerol sorbitan tetra-2-ethylhexylate; propylene glycol fatty acid esters such as propylene glycol monostearate; hydrogenated castor oil derivatives such as polyoxyethylene hydrogenated castor oil 40 (HCO-40), polyoxyethylene hydrogenated castor oil 50 (HCO-50), polyoxyethylene hydrogenated castor oil 60 (HCO-60), and polyoxyethylene hydrogenated castor oil 80; polyoxyethylene sorbitan fatty acid esters such as polyoxyethylene (20) sorbitan monolaurate (polysorbate 20), polyoxyethylene (20) sorbitan monostearate (polysorbate 60), polyoxyethylene (20) sorbitan monooleate (polysorbate 80), and polyoxyethylene (20) sorbitan isostearate; polyoxyethylene monococonut oil fatty acid glyceryl; glycerol alkyl ethers; alkyl glucosides; polyoxyalkylene alkyl ethers such as polyoxyethylene cetyl ether; amines such as stearylamine and oleylamine; silicone surfactants such as polyoxyethylene/methylpolysiloxane copolymers, lauryl PEG-9 polydimethylsiloxyethyl dimethicone, and PEG-9 polydimethylsiloxyethyl dimethicone; natural surfactants such as phospholipid, surfactin, and saponin; fatty acid amide amines such as stearamidoethyl diethylamine and stearamidopropyl diethylamine; alkylamines such as trilaurylamine, dimethylstearylamine, and di-2-ethylhexylamine; and betaine amphoteric surfactants such as stearamidopropyl dimethylamine and lauryl hydroxysulfobetaine.

Examples of the thickener include guar gum, locust bean gum, carrageenan, xanthan gum, polyvinyl alcohol, polyvinylpyrrolidone, carboxyvinyl polymers, acrylic acid alkyl methacrylate copolymers, polyethylene glycol, bentonite, alginic acid, macrogol, sodium chondroitin sulfate, hyaluronic acid, and cellulose thickeners (such as methyl cellulose, ethyl cellulose, hydroxyethyl cellulose, hydroxymethyl cellulose, hydroxypropyl cellulose, hydroxypropyl methyl cellulose, carboxymethyl cellulose, and carboxyethyl cellulose).

Examples of the antiseptic or preservative include benzoic acid, sodium benzoate, dehydroacetic acid, sodium dehydroacetate, isobutyl p-hydroxybenzoate, isopropyl p-hydroxybenzoate, butyl p-hydroxybenzoate, ethyl p-hydroxybenzoate, propyl p-hydroxybenzoate, benzyl p-hydroxybenzoate, methyl p-hydroxybenzoate, phenoxyethanol, benzyl alcohol, chlorobutanol, sorbic acid and salts thereof, chlorhexidine gluconate, alkanediol, and glycerol fatty acid esters. Some of the antiseptics or preservatives are included in the sterilizer (F).

Examples of the pH adjuster include inorganic acids (such as hydrochloric acid and sulfuric acid), organic acids (such as lactic acid, sodium lactate, citric acid, sodium citrate, succinic acid, and sodium succinate), inorganic bases (such as potassium hydroxide and sodium hydroxide), and organic bases (such as triethanolamine, diisopropanolamine, and triisopropanolamine).

Examples of the stabilizer include sodium polyacrylate, dibutylhydroxytoluene, and butylhydroxyanisole.

Examples of the chelating agent include disodium EDTA and calcium disodium EDTA.

Examples of the irritation reducing agent include glycyrrhiza extract and sodium alginate.

Examples of the ultraviolet absorber or ultraviolet scattering agent include 2-ethylhexyl p-methoxycinnamate, hexyl 2-[4-(diethylamino)-2-hydroxybenzoyl]benzoate, 2,4,6-tris[4-(2-ethylhexyloxycarbonyl)anilino]-1,3,5-triazine, t-butylmethoxydibenzoylmethane, ethylhexyl dibenzylidene dioxoimidazolidine propionate, ethylhexyl triazone, p-aminobenzoic acid and derivatives thereof, octyl p-dimethylaminobenzoate, ethylene glycol salicylate, dihydroxybenzophenone, titanium oxide, and zinc oxide.

Examples of the coloring agent include pigments described in Handbook of Legal Pigment (edited by Japan Cosmetic Industry Association (2004)).

The solid oily external composition of the present invention does not necessarily contain ingredients for concealing skin diseases such as skin inflammation, redness, and pigmentation.

Examples of the flavoring agent include various essential oils including herbal essential oils such as lavender oil, rosemary oil, clary sage oil, thyme oil, bergamot oil, and eucalyptus oil, mint essential oils such as peppermint oil, spearmint oil, and Japanese mint oil, and citrus essential oils such as orange oil, lemon oil, and grapefruit oil, and compound perfumes. Some of the flavoring agents are included in the ingredient (G).

Examples of the additional physiologically active or pharmacologically active ingredient (physiologically active or pharmacologically active ingredient other than the ingredients (A) to (G)) include a moisturizing ingredient, a vitamin, a peptide or derivatives thereof, a blood circulation accelerator, a cell activator, an antiaging ingredient, a keratin-softening ingredient, an astringent ingredient, an amino acid, a protein, a plant extract, a seaweed extract, an antifungal agent, and a whitening ingredient.

The additional physiologically active or pharmacologically active ingredients maybe used singly or in combination of two or more of them.

The additional physiologically active or pharmacologically active ingredient can be used in such a range as not to impair the effect of the invention.

Examples of the moisturizing ingredient include polyhydric alcohols such as glycerol, dipropylene glycol, 1,3-butylene glycol, propylene glycol, polyethylene glycol, diglycerol, pentanediol, hexanediol, and octanediol, saccharides such as trehalose, xylitol, and sorbitol, polymer compounds such as sodium hyaluronate, heparinoid, sodium chondroitin sulfate, keratin, chitin, and chitosan, lipids such as ceramide, cholesterol, and phospholipid, and plant extracts such as camomile extract, hamamelis extract, tea extract, and aloe extract.

Examples of the vitamins include vitamin Es such as dl-α-tocopherol, dl-α-tocopherol acetate, dl-α-tocopherol succinate, and dl-α-tocopherol calcium, succinate, ubiquinone derivatives and pharmaceutically or physiologically acceptable salts thereof, riboflavin, flavin mononucleotide, flavin-adenine dinucleotide, riboflavin butyrate, riboflavin tetrabutyrate, sodium riboflavin 5′-phosphate, riboflavin tetranicotinate, dl-α-tocopherol nicotinate, benzyl nicotinate, methyl nicotinate, β-butoxyethyl nicotinate, 1-(4-methylphenyl)ethyl nicotinate, ascorbigen-A, ascorbyl stearate, ascorbyl palmitate, L-ascorbyl dipalmitate, methylhesperidin, ergocalciferol, cholecalciferol, phylloquinone, farnoquinone, γ-oryzanol, dibenzoylthiamine, dibenzoylthiamine hydrochloride, thiamine hydrochloride, thiamine cetyl hydrochloride, thiamine thiocyanate, thiamine lauryl hydrochloride, thiamine nitrate, thiamine monophosphate, thiamine lysine salt, thiamine triphosphate, thiamine monophosphate phosphate salt, thiamine monophosphate, thiamine diphosphate, thiamine diphosphate hydrochloride, thiamine triphosphate, thiamine triphosphate monophosphate salt, pyridoxine hydrochloride, pyridoxine acetate, pyridoxal hydrochloride, pyridoxal 5′-phosphate, pyridoxamine hydrochloride, cyanocobalamin, hydroxocobalamin, deoxyadenosylcobalamin, folic acid, pteroylglutamic acid, nicotinic acid, nicotinic acid amide, pantothenic acids such as pantothenic acid, calcium pantothenate, pantothenyl alcohol (panthenol), D-pantesine, D-pantethine, coenzyme A, and pantothenyl ethyl ether, biotin, biocytin, ascorbic acid, sodium ascorbate, dehydroascorbic acid, sodium ascorbate phosphate, magnesium ascorbate phosphate, carnitine, ferulic acid, α-lipoic acid, orotic acid, hesperidin, γ-oryzanol, orotic acid, rutin, and eriocitrin.

Examples of the peptide or derivatives thereof include keratin degradation peptide, hydrolyzed keratin, collagen, gelatin, elastin, elastin degradation peptide, collagen degradation peptide, hydrolyzed collagen, and hydrolyzed silk.

Examples of the blood circulation accelerator preferably include plant-derived ingredients. Examples include ingredients derived from Panax ginseng, Angelica keiskei, arnica, ginkgo, Isodon japonicus, Nasturtium officinale, carrot, gentian, burdock root, rice, hawthorn, shiitake mushrooms, Crataegus laevigata, Juniperus communis, Cnidium rhizome, Swertia japonica, thyme, clove, Citrus unshiu peel, Angelica root, peach kernel, bitter orange peel, carrot, garlic, Ruscus aculeatus, grape, Paeonia suffruticosa, horse chestnut, Melissa officinalis, Citrus junos, Coix seed, rosemary, rose hip, peach, apricot, walnut, corn, and the like (including extracts of these plants) and glycosyl hesperidin.

Examples of the cell activator include amino acids such as γ-aminobutyric acid, γ-amino-β-hydroxybutyric acid, and ε-aminocaproic acid, vitamins such as retinol, thiamine, riboflavin, pyridoxine hydrochloride, pantothenic acids, and biotin, α-hydroxy acids such as glycolic acid and lactic acid, tannins, flavonoids, saponins, allantoin, Kankoh-so 301, placenta extract, hinokitiol , cepharanthin, and kiwi seed extract.

Examples of the antiaging ingredient include pangamic acid, kinetin, ursolic acid, turmeric extract, sphingosine derivatives, silicon, silicic acid, N-methyl-L-serine, and mevalonolactone.

Examples of the keratin-softening ingredient include salicylic acid, salicylic acid derivatives, glycolic acid, fruit acid, phytic acid, sulfur, ethyl alcohol, isopropyl alcohol, propanol, butanol, 1,3-butylene glycol, propylene glycol, polyethylene glycol (macrogol), glycerol, benzyl alcohol, phenylethyl alcohol, propylene carbonate, hexyldodecanol, allantoin, dimethyl sulfoxide, dimethylacetamide, dimethylformamide, triethanolamine, diisopropvl adipate, ethyl laurvlate, lanolin, fatty acid dialkylolamide, urea, sulfur, resorcin, glycolic acid, phytic acid, lactic acid, lactate, sodium hydroxide, and potassium hydroxide.

Examples of the astringent ingredient include zinc p-phenolsulfonate, zinc oxide, menthol, and ethanol.

Examples of the plant extract include extracts of plants such as Mulberry bark, Saxifraga stolonifera, Perilla frutescens, rice bran, sake lees, white mustard, Paeonia lactiflora, Callicarpa japonica, lotus seeds, Coix seeds, Pandanus amaryllifolius Roxb, Arcangelicia flava Merrilli, camomile, Salicornia europaea, rice leaves, apricot fruit, Betaphycus gelatinum, rose flower, bamboo shoot skin, gentian, carrot, Panax ginseng, red ginseng, Luffa cylindrica, peach, peach kernel, Actinidia deliciosa, sunflower, Zizyphus joazeiro, Handroanthus impetiginosus, daylily, hibiscus flower, Alchemilla monis, cherimoya, mango, Benifuki, Bletilla striata, Zanthoxylum fruit skin or seed coat, Carthamus tinctorius flower, Casa Blanca lily, Psidium guajava leaves, Houttuynia cordata, Citrus maxima, aloe fig flower, apple, white asparagus, mate, cherry leaves, and ylang ylang leaves.

Examples of the seaweed extract include green algae such as Chlorella vulgaris, Chlorella pyrenoidosa, Chlorella ellipsoidea, green laver, sea lettuce, and Ulva pertusa; brown algae such as kelps (such as Saccharina sculpera, Saccharina japonica var. japonica, Saccharina japonica var. ochotensis, Saccharina japonica var. religiosa, Saccharina angustata), giant kelp, brown seaweed or green seaweed, Nemacystus decipiens, Undaria undarioides, Hizikia fusiformis, Fucus evanescens, Anthogorgia bocki, Padina australis, Padina australis var. cuneata, Padina boryana, Padina crassa, Padina japonica, Padina minor, and Padina stipitata; and red algae such as Grateloupia sparsa, Gelidium elegans or Gelidium amansii, Ptilophora subcostata, Gelidium japonicum, Pterocladia tenuis, Pterocladiella capillacea, Yatabella hirsuta, Acanthopeltis japonica, Gelidiella acerosa, Meristotheca papulosa, Eucheuma serra, Eucheuma amakusaense, Eucheuma muricatum, Eucheuma arnoldii, Chondrus ocellatus, Chondrus giganteus, Chondrus crispus, Chondrus yendoi, Chondrus armatus, Chondrus pinnulatus, Chondrus elatus, Chondrus verrucosus, Chondrus nipponicus, Chondrus pinnulatus, Gigartina tenella, Chondracanthus teedei, Chondracanthus intermedius, Acrosorium flabellatum, Acrosorium venulosum, Acrosorium polyneurum, Acrosorium yendoi, and akamomijinori.

Examples of the antifungal agent include terbinafine, naftifine, butenafine, tolnaftate, liranaftate, miconazole, lanoconazole, luliconazole, isoconazole, ketoconazole, clotrimazole, neticonazole, sulconazole, bifonazole, oxiconazole, econazole, fluconazole, itraconazole, fosfluconazole, voriconazole, efinaconazole, butoconazole, fenticonazole, and sertaconazole.

Examples of the whitening ingredient include tocopherol, ascorbic acid, tranexamic acid, arbutin, 4-alkylresorcinol, 4-methoxysalicylic acid, hydroquinone, kojic acid, salts thereof or derivatives thereof, placenta extract, Phellodendron bark extract, Saxifraga stolonifera extract, and aloe extract.

### Base Material or Carrier

Examples of the base material or carrier include an oily base and an aqueous base.

Examples of the oily base include hydrocarbons such as petroleum (white petrolatum and yellow petrolatum), gelled hydrocarbons (such as plastibase), ozokerite, ceresin, microcrystalline wax, squalene, squalane, α-olefin oligomer, paraffin, liquid paraffin, and light liquid paraffin; higher alcohols such as cetanol, cetostearyl alcohol, stearyl alcohol, and behenyl alcohol; sterols such as cholesterol, phytosterol, and phytosteryl hydroxystearate; vegetable tallows such as shea butter, carnauba wax, cacao butter, and candelilla wax; vegetable oils such as avocado oil, olive oil, camellia oil, macadamia nut oil, evening primrose oil, jojoba oil, rape seed oil, egg yolk oil, sesame oil, castor oil, safflower oil, cottonseed oil, soybean oil, tea seed oil, rice bran oil, rice germ oil, wheat germ oil, peanut oil, sunflower oil, almond oil, corn oil, coconut oil, orange oil, sage oil, palm oil, mink oil, meadowfoam oil, lavender oil, rosemary oil, and rose hip oil; animal oils and fats such as lanolin, orange roughy oil, squalane, horse fat, spermaceti wax, and beeswax; hardened oils; silicone oils such as methylpolysiloxane, cross-linked methylpolysiloxane, highly polymerized methylpolysiloxane, cyclic silicone, alkyl-modified silicone, cross-linked alkyl-modified silicone, amino-modified silicone, polyether-modified silicone, polyglycerol-modified silicone, cross-linked polyether-modified silicone, cross-linked alkyl polyether-modified silicone, silicone/alkyl chain-co-modified polyether-modified silicone, silicone/alkyl chain-co-modified polyglycerol-modified silicone, polyether-modified branched silicone, polyglycerol-modified branched silicone, acrylic silicon, phenyl-modified silicone, and silicone resins; natural polymer derivatives such as ethyl cellulose, hydroxypropyl cellulose, hydroxypropyl methyl cellulose, cationized guar gum, and acetylated hyaluronic acid; synthetic polymers such as polyvinylpyrrolidone, carboxyvinyl polymers, and acrylic acid alkyl methacrylate copolymers; natural polymers such as carrageenan, alginic acid, cellulose, guar gum, quince seeds, dextran, gellan gum, and hyaluronic acid; esters such as isopropyl myristate, octyldodecyl myristate, isopropyl palmitate, cetyl palmitate, isononyl isononanoate, pentaerythrite tetra-2-ethylhexanoate, and glyceryl tri(caprylate/caprate); polysaccharides such as dextrin and maltodextrin; and glycol ethers such as ethylene glycol monomethyl ether, ethylene glycol monoethyl ether, ethylene glycol monopropyl ether, diethylene glycol monomethyl ether, diethylene glycol monoethyl ether, diethylene glycol monopropyl ether, diethylene glycol monobutyl ether, propylene glycol monoethyl ether, propylene glycol monopropyl ether, dipropylene glycol monoethyl ether, and dipropylene glycol monopropyl ether.

Examples of the aqueous base include, in addition to water and buffers, lower alcohols such as ethanol and isopropanol; and polyhydric alcohols such as polyethylene glycol, propylene glycol, dipropylene glycol, 1,3-butylene glycol, glycerol, diglycerol, and isoprene glycol.

The base materials or carriers may be used singly or in combination of two or more of them.

Of them, hydrocarbon base materials are preferred, and petroleum, paraffin, liquid paraffin, ceresin, microcrystalline wax, squalene, squalane, α-olefin oligomer, and light liquid paraffin are preferred. When hydrocarbon base materials are contained, the total content thereof is preferably 40% by weight or more, particularly preferably 50% by weight or more, and specifically preferably 60% by weight or more relative to the total amount of the base material or carrier. The total content can be 80% by weight or more or 90% by weight or more. The base material can be only the hydrocarbon base material (at a content of 100% by weight relative to the total amount of the base material or carrier).

A base material containing a hydrocarbon compound and an aqueous base is also preferred. In this case, the content of the aqueous base can be, for example, 15% by weight or less, particularly 10% by weight or less, and specifically 5% by weight or less relative to the total amount of the solid oily external composition. The content can be 3% by weight or less. The content of the aqueous base can be 0.00001% by weight or more relative to the total amount of the solid oily external composition.

Specifically, a base material containing a hydrocarbon compound and water is also preferred. In this case, the content of water can be, for example, 30% by weight or less, particularly 20% by weight or less, specifically 10% by weight or less, specifically 5% by weight or less (especially less than 5% by weight), specifically 3% by weight or less, specifically 1% by weight or less, and specifically 0.5% by weight or less relative to the total amount of the solid oily external composition. The content of water can be 0.00001% by weight or more relative to the total amount of the solid oily external composition.

A base material containing a hydrocarbon compound and a hardened oil is also preferred. In this case, the total content of the hydrocarbon base material is preferably 40% by weight or more, particularly preferably 50% by weight or more, and specifically preferably 60% by weight or more relative to the total amount of the base material or carrier. The total content can be 80% by weight or more or 90% by weight or more. In this case, the upper limit of the total content of the hydrocarbon base material can be about 99% by weight relative to the total amount of the base material or carrier.

When the solid oily external composition of the present invention contains a water-soluble polyhydric alcohol (polyhydric alcohols are typically water-soluble), the content thereof can be less than 25% by weight, particularly less than 10% by weight, and specifically less than 5% by weight relative to the total amount of the composition. The composition can contain no water-soluble polyhydric alcohol.

The solid oily external composition of the present invention can be a composition other than “compositions containing at least one pharmaceutically active ingredient selected from adrenal cortex hormones and antihistamines, a water-soluble polyhydric alcohol, a higher fatty acid salt, a water-soluble polymer, and purified water”. The solid oily external composition of the present invention can be a composition other than “compositions containing a water-soluble polyhydric alcohol, a higher fatty acid salt, a water-soluble polymer, and purified water”.

The solid oily external composition of the present invention can be a composition other than “compositions containing a steroid, an unsaturated fatty acid alcohol, an alkylene glycol selected from propylene glycol, butylene glycol, dipropylene glycol, and dibutylene glycol, and a rigidity improving agent (particularly a thickener, specifically a wax-like substance, and more specifically a monoester of a carboxylic acid having 14 to 36 carbon atoms and an alcohol having 14 to 36 carbon atoms, a triglyceride of fatty acids having 18 to 36 carbon atoms, or a glycol ester of fatty acids having 18 to 36 carbon atoms)”. The solid oily external composition of the present invention can be a composition other than “compositions containing an unsaturated fatty acid alcohol, an alkylene glycol selected from propylene glycol, butylene glycol, dipropylene glycol, and dibutylene glycol, and a rigidity improving agent (particularly a thickener, specifically a wax-like substance, and more specifically a monoester of a carboxylic acid having 14 to 36 carbon atoms and an alcohol having 14 to 36 carbon atoms, a triglyceride of fatty acids having 18 to 36 carbon atoms, or a glycol ester of fatty acids having 18 to 36 carbon atoms)”.

The solid oily external composition of the present invention can be a composition other than “compositions containing a corticosteroid, petroleum, wax, about 5 to 20% by weight of propylene glycol, and an emulsifier and containing substantially no water”. The solid oily external composition of the present invention can be a composition other than “compositions containing petroleum, wax, about 5 to 20% by weight of propylene glycol, and an emulsifier and containing substantially no water”.

### Oily Composition

The solid oily external composition of the present invention is an oily composition in which the base material or carrier is mainly an oily base. In the composition of the present invention, the content of the aqueous base is preferably 15% by weight or less, more preferably 10% by weight or less, even more preferably 5% by weight or less, and still more preferably 3% by weight or less relative to the total amount of the composition. The base material or carrier may consist of substantially only an oily base (specifically consist of only an oily base). When the solid oily external composition of the present invention contains an aqueous base, the content of the aqueous base can be 0.00001% by weight or more relative to the total amount of the composition.

In particular, the content of water in the composition of the present invention is preferably 30% by weight or less, more preferably 20% by weight or less, even more preferably 10% by weight or less, specifically preferably 5% by weight or less, still more preferably less than 5% by weight, and most preferably 3% by weight or less relative to the total amount of the composition. The content can be 1% by weight or less and specifically 0.5% by weight or less. The composition may contain substantially no water (specifically contain no water). When the solid oily external composition of the present invention contains water, the content of water can be 0.00001% by weight or more relative to the total amount of the composition.

When the solid oily external composition of the present invention contains an aqueous base, particularly water, the composition is typically a water-in-oil emulsion but can be an oil-in-water emulsion depending on a surfactant used.

### Properties

The solid oily external composition of the present invention is a solid composition at a temperature of 25° C. and can be specifically a molded composition. The “molded” means that a composition maintains a constant shape without flowing at a temperature of 25° C. The “molded composition” also includes a composition having such plasticity as to be deformable by application of force and a gel-like composition.

Specifically, a composition molded into a stick shape or a rod shape is preferred. The “stick shape” and “rod shape” include a shape having a larger length in the axis direction than the diameter in the circumferential direction and a shape having a smaller length in the axis direction than the diameter in the circumferential direction. The cross-sectional shape in the circumferential direction is not specifically limited, and examples include a round, an ellipse, a polygonal, and an indefinite shape. The end face (upper end face and/or lower end face) in the axis direction may be a flat face, a convex face, or a concave face.

The hardness of the solid oily external composition of the present invention can be, for example, 1 to 100 (g) and is preferably 5 to 90 (g), more preferably 10 to 80 (g), even more preferably 15 to 65 (g), and specifically preferably 20 to 50 (g). When the hardness is in such a range as above, an appropriate amount of the composition can be applied without excess irritation onto an application area, the composition is prevented from breaking at the time of use (particularly from breaking when an oily composition packed in a feeding container is fed), and the composition has satisfactory smoothness.

In the present invention, the hardness is determined as follows: a drug product packed in a cylindrical container is cooled at a constant temperature of about 25° C. for 12 hours or more; a rheometer (trade name “SUN RHEO METER CR-100”, manufactured by Sun Scientific Co., Ltd.) is used, and a cylindrical adapter (adapter) having a diameter of 1 mm is placed at the center of the cylindrical container and is advanced at a speed of 20 mm/min; and the maximum value for 30 seconds from the start of advancing is determined as the hardness (unit: g).

### Application Site

The solid oily external composition of the present invention can be applied onto a skin or a mucous membrane and is particularly preferably a skin external composition. The skin includes the scalp.

### Production Method

The solid oily external composition of the present invention can be produced in a usual manner. For example, all the ingredients (including solid ingredients and/or liquid or flowable ingredients) are mixed and stirred while heated to be dissolved or dispersed, then the mixture is packed in a mold and then cooled, and the cooled mixture is packed in a container, giving a solid oily external composition. The production method is not limited to this procedure.

### Friction Reduction Method

The present invention encompasses a method of reducing a friction of a solid oily external composition against a skin or a mucous membrane when the composition is applied, and the method includes adding (A) a steroid compound and/or (B) an antihistamine to the composition. The types and the contents of the ingredients, the properties of the composition, and the like are as described above for the solid oily external composition of the present invention.

### EXAMPLES

The present invention will next be described in more detail with reference to examples, but the present invention is not limited thereto.

### Test Example 1

### Friction Evaluation at the Time of Application (1)

A stick-shaped oily composition (a weight of about 3.4 g) having a formulation shown in Table 1 was prepared by packing the composition in a feeding container having a diameter of 1.3 cm in a usual manner. Specifically, ingredients shown in Table 1 were heated and dissolved; then the mixture was packed in a cylinder of a feeding container, next was cooled to be solidified into a rod shape, and then was dissolved and solidified again; and a drug product having a flat surface was prepared.

The friction of each stick drug product at the time of application was evaluated by using a friction tester (“Static-Dynamic Friction Measuring Device (TriLab TriboMaster Type: TL201Sa)” manufactured by Trinity-Lab Inc.).

Specifically, an artificial leather cut into 5 cm in length and 15 cm in width was attached to the friction tester. Next, each stick-shaped composition packed in a container was fed by about 0.5 cm from the container and set to a holder, and the average dynamic friction coefficient (μk) was determined in conditions of a conveyance speed of 2 mm/min, a measurement distance of 50 mm, and a load of 50 g.

In accordance with Equation (1), the ratio of the friction coefficient of each composition of Examples was calculated where the friction coefficient of the composition of Comparative Example 1 containing neither the steroid compound (A) nor the antihistamine (B) was 100.

Friction coefficient ratio (%)=(friction coefficient of Example/friction coefficient of Comparative Example 1)×100 (1)

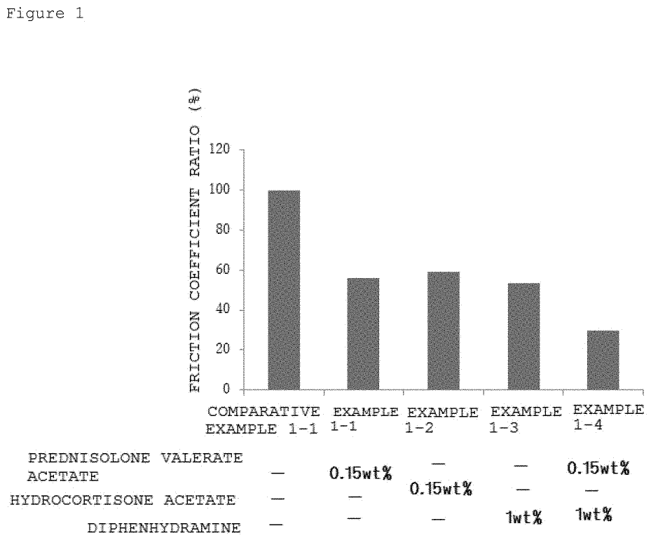

The friction coefficient ratios are shown in Table 1 and FIG. 1.

TABLE 1
(unit: % by weight)

| | | Exam- | Exam- | Exam- | Exam- |
|---|---|---|---|---|---|
| | Comparative | ple | ple | ple | ple |
| | Example 1-1 | 1-1 | 1-2 | 1-3 | 1-4 |
| Prednisolone | — | 0.15 | — | — | 0.15 |
| valerate | | | | | |
| acetate (PVA) | | | | | |
| Hydrocortisone | — | — | 0.15 | — | — |
| acetate | | | | | |
| Diphenhydramine | — | — | — | 1 | 1 |
| White | 27 | 27 | 27 | 27 | 27 |
| petrolatum | | | | | |
| Liquid paraffin | 46 | 45.85 | 45.85 | 45 | 44.85 |
| Hardened oil | 4 | 4 | 4 | 4 | 4 |
| Paraffin | 20 | 20 | 20 | 20 | 20 |
| Crotamiton | 3 | 3 | 3 | 3 | 3 |
| Total amount | 100 g | 100 g | 100 g | 100 g | 100 g |
| Friction | 100 | 56.2 | 59.2 | 53.4 | 29.7 |
| coefficient | | | | | |
| ratio (%) | | | | | |

* The used hardened oil was a hydrogenated castor oil type.

As shown in Table 1, adding prednisolone valerate acetate, hydrocortisone acetate, or diphenhydramine to the oily base lowered friction coefficient, and adding prednisolone valerate acetate and diphenhydramine to the oily base markedly lowered friction coefficient.

The hardness of the oily composition in Example 1-1 was determined to be 32.63 (g).

The hardness was determined as follows: a drug product separately prepared in a similar manner to that in Example 1-1 was allowed to stand at a constant temperature of about 25° C. for 12 hours or more; a rheometer (trade name “SUN RHEO METER CR-100”, manufactured by Sun Scientific Co., Ltd.) was used, and a cylindrical adapter (adapter) having a diameter of 1 mm was placed at the center of the feeding container filled with the oily composition and was advanced at a speed of 20 mm/min; and the maximum value for 30 seconds from the start of advancing was determined as the hardness (unit: g).

### Test Example 2

### Friction Evaluation at the Time of Application (2)

Friction evaluation at the time of application was performed in a similar manner to that in Test Example 1 except that a stick-shaped oily composition having a formulation shown in Table 2 was used.

In accordance with Equation (2), the ratio of the friction coefficient of the composition of Example 2-1 was calculated where the friction coefficient of the composition of Comparative Example 2-1 was 100.

Friction coefficient ratio (%)=(friction coefficient of Example 2-1/friction coefficient of Comparative Example 2-1)×100 (2)

The friction coefficient ratios are shown in Table 2.

TABLE 2
(unit: % by weight)

| | Comparative | |
|---|---|---|
| | Example 2-1 | Example 2-1 |
| Prednisolone valerate acetate | — | 0.15 |
| White petrolatum | 27 | 27 |
| Liquid paraffin | 45.99 | 45.84 |
| Hardened oil | 4 | 4 |
| Paraffin | 20 | 20 |
| Purified water | 0.01 | 0.01 |
| Propylene glycol | 3 | 3 |
| Total amount | 100 g | 100 g |
| Friction coefficient ratio (%) | 100 | 57.2 |

* The used hardened oil was a hydrogenated castor oil type.

As shown in Table 2, it was ascertained that a composition containing water and an aqueous base as the base material also had a lower friction coefficient, and the friction when the stick composition was applied was reduced.

### Test Example 3

### Friction Evaluation at the Time of Application (3)

A stick-shaped oily composition containing hydrocortisone as the steroid compound (A) was prepared, and friction evaluation at the time of application was performed in a similar manner to that in Test Example 1. It was ascertained that the friction coefficient was reduced by addition of hydrocortisone, and the friction when the stick composition was applied was reduced.

### Preparation Example

In accordance with the following formulations, solid oily external compositions (Drug Product Formulation Examples 1 to 3) were prepared.

<Drug Product Formulation Example 1>

| (1) Liquid paraffin | balance | |
|---|---|---|
| (2) Squalane | 10.0% | by weight |
| (3) White petrolatum | 25.0% | by weight |
| (4) Paraffin | 20.0% | by weight |
| (5) Dexamethasone acetate | 0.025% | by weight |
| (6) Hardened oil (hydrogenated castor oil type) | 5.0% | by weight |
| (7) Crotamiton | 3.0% | by weight |
| Total amount | 100 | g |

<Drug Product Formulation Example 2>

| (1) Liquid paraffin | balance | |
|---|---|---|
| (2) α-Olefin oligomer | 10.0% | by weight |
| (3) White petrolatum | 25.0% | by weight |
| (4) Paraffin | 20.0% | by weight |
| (5) Prednisolone valerate acetate | 0.15% | by weight |
| (6) Hardened oil (hardened rape seed oil type) | 5.0% | by weight |
| (7) Crotamiton | 3.0% | by weight |
| Total amount | 100 | g |

<Drug Product Formulation Example 3>

| (1) Liquid paraffin | balance | |
|---|---|---|
| (2) White petrolatum | 27.0% | by weight |
| (3) Hardened oil | 4.0% | by weight |
| (4) Paraffin | 20.0% | by weight |
| (5) Diphenhydramine | 1.0% | by weight |
| Total amount | 100 | g |

### INDUSTRIAL APPLICABILITY

The solid oily external composition of the present invention is smoothly applicable onto a skin or a mucous membrane and is comfortable to use.

1. A solid oily external composition comprising:
at least one selected from the group consisting of (A) a steroid compound and (B) an antihistamine,
wherein the steroid compound (A) is a compound selected from the group consisting of prednisolone, dexamethasone, hydrocortisone, esters thereof, and salts thereof, wherein the antihistamine (B) is a compound selected from the group consisting of diphenhydramine and salts thereof.

2. (canceled)

3. The composition according to claim 1, wherein the steroid compound (A) is a compound selected from the group consisting of prednisolone valerate acetate, dexamethasone acetate, hydrocortisone, and hydrocortisone acetate.

4. (canceled)

5. The composition according to claim 1, wherein the steroid compound (A) is contained at a content of 0.0001 to 5% by weight relative to a total amount of the composition.

6. The composition according to claim 1, wherein the antihistamine (B) is contained at a content of 0.01 to 5% by weight relative to a total amount of the composition.

7. The composition according to claim 1, further comprising at least one selected from the group consisting of (C) an antipruritic other than the antihistamine (D) a local anesthetic, (E) an anti-inflammatory agent, (F) a sterilizer, and (G) a refreshing agent.

8. The composition according to claim 1, wherein the composition is a skin external composition.

9. The composition according to claim 1, wherein the composition has a stick shape.

10. A method of reducing a friction of a solid oily external composition against a skin or a mucous membrane when the composition is applied, the method comprising:
adding (A) a steroid compound and/or (B) an antihistamine to the composition, wherein the steroid compound (A) is a compound selected from the group consisting of prednisolone, dexamethasone, hydrocortisone, esters thereof, and salts thereof, wherein the antihistamine (B) is a compound selected from the group consisting of diphenhydramine and salts thereof.
